(19) United States
(12) Patent Application Publication
(10) Pub. No.: US 2024/0308958 A1
(43) Pub. Date: Sep. 19, 2024

(54) SYNTHETIC PREPARATION FOR DIROXIMEL FUMARATE

(71) Applicant: BIOGEN MA INC., Cambridge, MA (US)

(72) Inventors: Daw-long Albert KWOK, Cambridge, MA (US); Chao Fei, Cambridge, MA (US); Erwin Irdam, Cambridge, MA (US); Donald Walker, Cambridge, MA (US); Markus Grohmann, Dottikon (CH); Jens Mohr, Dottikon (CH); Janina Bucher, Dottikon (CH)

(21) Appl. No.: 18/571,090

(22) PCT Filed: Jun. 14, 2022
(86) PCT No.: PCT/US2022/033409

§ 371 Date: Dec. 15, 2023

Related U.S. Application Data
(60) Provisional application No. 63/210,660, filed on Jun. 15, 2021.

(51) Int. Cl. C07D 207/404

(52) U.S. Cl. CPC C07D 207/404

(57) ABSTRACT

Disclosed is a method of preparing diroximel fumarate represented by the following structural formula (I) The method comprises reacting ethy lene carbonate with succinimide to form a hydroxyethyl succinimide intermediate; and reacting the intermediate with monomethyl fumarate to form the product compound.

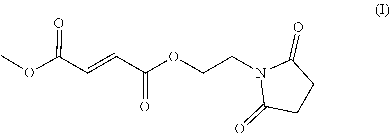 [SMILES: COC(=O)/C=C/C(=O)OCCN1C(=O)CCC1=O]

### RELATED APPLICATIONS

This application claims the benefit of U.S. Provisional Application Ser. No. 63/210,660, filed Jun. 15, 2021, the entire teachings of which are incorporated herein by reference.

### FIELD OF THE INVENTION

Disclosed is an improved method for preparing diroximel fumarate. The method comprises reacting ethylene carbonate with succinimide to form a hydroxyethyl succinimide intermediate; and reacting the intermediate with monomethyl fumarate to form diroximel fumarate.

### BACKGROUND OF THE INVENTION

Diroximel fumarate is sold under the brand name Vumerity and is a medication used for the treatment of relapsing forms of multiple sclerosis. Diroximel fumarate was first disclosed in U.S. Pat. No. 8,669,281 and approved for medical use in the United States in October 2019.

Successful development of a new drug requires a cost efficient, high yield synthesis that is amendable to large scale manufacture. U.S. Pat. No. 8,669,281 discloses preparing diroximel fumarate by reacting monomethyl fumarate with hydroxyethyl succinimide in the presence of the coupling agent 2-(1H-benzotriazole-1-yl)-1,1,3,3-tetramethylaminium tetrafluoroborate (hereinafter “TBTU”) as follows:

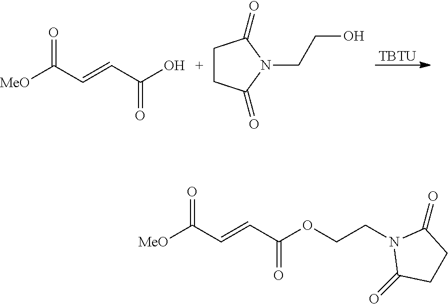

(this reaction is hereinafter to referred to herein as the “Coupling Reaction”). However, U.S. Pat. No. 8,669,281 reports a yield of only 35% for this step. Therefore, the preparation of diroximel furmarate using the Coupling Reaction would necessarily require adding additional reaction steps for preparing hydroxyethyl succinimide. As such, improved methodology for preparing diroximel fumarate is needed.

### SUMMARY OF THE INVENTION

It has now been found that replacing the TBTU coupling agent with 1-ethyl-3-(3-dimethylaminopropyl)carbodiimide (hereinafter “EDC”) increases the yield of the Coupling Reaction to 91% (see Examples 5 and 6). It has also been found that a reaction preparing the hydroxyethyl succinimide starting material from ethylene carbonate and succinimide can be combined in “one pot” with the Coupling Reaction. The reaction sequence of this one pot synthesis of diroximel fumarate is shown schematically below:

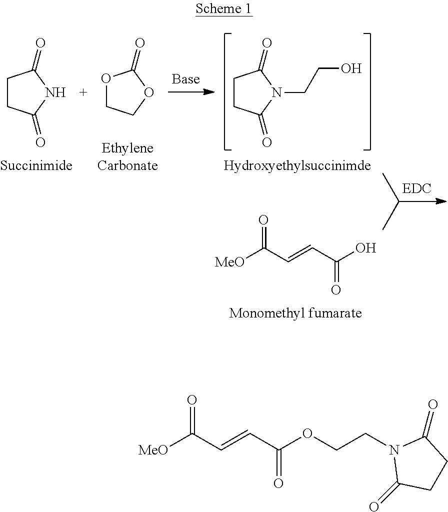

This preparation proceeds in a high overall yield (84%) and represents a substantial improvement over other processes used to prepare diroximel fumarate (Example 6). The process is also efficient in terms of its utilization of manufacturing equipment and solvent, given that it can be carried out without isolation of hydroxyethyl succinimide. Additionally, the efficiency of the process is high in terms of its scalability and reaction times.

Improvements have also found in the synthesis of diroximel fumarate shown in Scheme 2 below. These improvements result in increased yield, reduced cycle time and reduced levels of impurity formation:

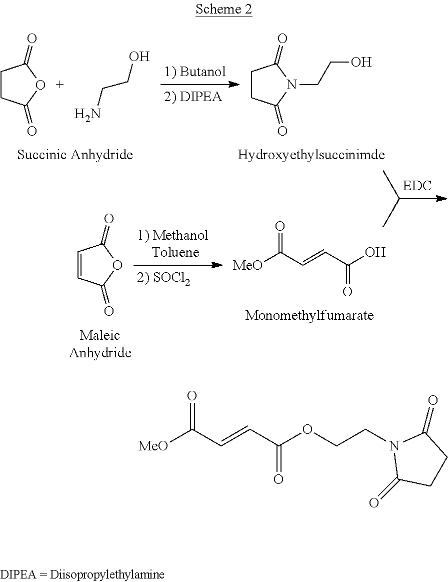

Specifically, use of between 0.02 and 0.10 equivalents of thionyl chloride relative to the maleic acid starting material in the preparation of monomethyl fumarate reduces the formation of undesirable fumaric acid by-product, thereby eliminating the need for recrystallization (Examples 1 and 2); use of between 0.1 and 0.3 equivalents of acetic acid relative to succinimide in the preparation of hydroxyethyl succinimide reduces the ring closure time from seventy-two to thirty-nine hours (Examples 3 and 4); and use of EDC as the coupling agent in the preparation of diroximel fumarate increases the yield to 92% (Example 5). All of these improvements are amendable to scale-up. Based on these discoveries, improved methods of preparing diroximel fumarate are disclosed herein.

One embodiment of the invention is method of preparing diroximel fumarate. The method comprises reacting ethylene carbonate with succinimide to form hydroxyethyl succinimide and then reacting hydroxyethyl succinimide with monomethyl fumarate to form diroximel fumarate. In one aspect, the two reactions are carried out in one pot, i.e., without isolating hydroxyethyl succinimide. In another aspect, the reaction is carried out in the presence of N-(3-dimethylaminopropyl)-N′-ethylcarbodiimide or a salt thereof, e.g., N-(3-dimethylaminopropyl)-N′-ethylcarbodiimide hydrochloride, and a basic catalyst.

Another embodiment of the invention is a method of preparing monomethyl fumarate. The method comprises the steps of: a) reacting methanol and maleic anhydride to form an intermediate product represented by

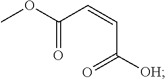

and b) reacting the intermediate product with a catalytic amount of thionyl chloride to form the monomethyl fumarate.

Yet another embodiment of the invention is a method of preparing hydroxyethyl succinimide. The method comprises reacting succinic anhydride with hydroxyethylamine to form an intermediate represented by the following structural formula:

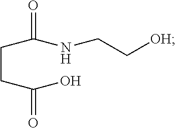 [SMILES: O=C(O)CCC(=O)NCCO]

and then reacting the intermediate with a non-nucleophilic amine base, such as diisopropylethylamine, preferably without isolation of the intermediate, to form hydroxyethyl succinimide. Acetic acid is preferably added (e.g., between 0.1 and 0.3, preferably between 0.1 and 0.2 equivalents of acetic acid relative to succinic anhydride) to the reaction between the intermediate and the non-nucleophilic amine base to increase the reaction rate.

Yet another embodiment of the invention is a method of preparing diroximel fumarate. The method comprises reacting hydroxyethyl succinimide with monomethyl fumarate in the presence of N-(3-dimethylaminopropyl)-N′-ethylcarbodiimide or a salt thereof, e.g., N-(3-dimethylaminopropyl)-N′-ethylcarbodiimide hydrochloride, and a basic catalyst to form diroximel fumarate.

### DETAILED DESCRIPTION OF THE INVENTION

The invention is directed to improved processes for preparing diroximel fumarate. In one process, succinimide and ethylene carbonate are reacted to form hydroxyethyl succinimide as an intermediate; and then reacting the hydroxyethyl succinimide intermediate with monomethyl fumarate. The two step sequence can be carried out in “one pot”, i.e., without isolating hydroxyethyl succinimide.

The reaction between hydroxyethyl succinimide and monomethyl fumarate is in one aspect carried out in the presence of a carboxylic acid coupling agent. A “carboxylic acid coupling reagent” activates the hydroxyl group of a carboxylic acid towards nucleophilic substitution by, for example, an alcohol, such as the alcohol group of hydroxyethyl succinimide. Carboxylic acid coupling reagents are known in the art and include e.g., carbodiimides, phosphonium reagents, aminium/uranium-imonium reagents, N-ethoxycarbonyl-2-ethoxy-1,2-dihydroquinoline, 2-propanephosphonic acid anhydride, 4-(4,6-dimethoxy-1,3,5-triazin-2-yl)-4-methylmorpholinium salt, bis-trichloromethylcarbonate, 1,1′-carbonyldiimidazole, mesyl chloride, propylphosphonic anhydride, pivaloyl chloride, oxalyl chloride and thionyl chloride. In one aspect, the coupling reagent is 1-ethyl-3-(3-dimethylaminopropyl)carbodiimide or a salt thereof, c.g., 1-ethyl-3-(3-dimethylaminopropyl)carbodiimide hydrochloride. Suitable solvents for this reaction would be apparent to one of skill in the art and include ketone solvents such as acetone and methyl ethyl ketone; ethereal solvents such as diethyl ether, di-tert-butyl ether, diisopropyl ether, 1,4-dioxane, dimethoxy ethane, dimethoxy methane, diglyme, ethyl tert-butyl ether, methyl tert-butyl ether, tetrahydrofuran, tetrahydropyran, and the like; and aprotic polar solvents such as acetonitrile. In one aspect, the solvent used is acetone.

The reaction between hydroxyethyl succinimide and monomethyl fumarate is in one aspect carried out in the presence of a carboxylic acid coupling agent and a basic catalysts. Suitable basic catalysts are those that are “non-productive, i.e., do not otherwise interfere with the reaction or cause side reactions. Examples of suitable basic catalysts include dimethylaminopyridine, 1-methylimidazole and triethylamine.

The reaction between succinimide and ethylene carbonate is in one aspect carried out in the presence of a non-nucleophilic amine base. In one aspect, a catalytic amount of the non-nucleophilic base is used. A “non-nucloephilic amine base” is a tertiary amine, an amine with one or two adjacent di or tri substituted carbon atoms or an amine in which the amine is otherwise stereochemically hindered by other nearby functional groups on the molecule. Examples include N,N-diisopropylethylamine, tetramethylpiperidine, 1,8-diazabicycloundec-7-ene, 2,6-di-tert-butylpyridine, 2,6-lutidine, dimethylaminopyridine, and pyridine. In one aspect, the non-nucleophilic amine base is 1,8-diazabicycloundec-7-ene (DBU).

The reaction between succinimide and ethylene carbonate in one aspect can be carried out neat, i.e., without a solvent. In another aspect, small amounts of solvent can be added to the reaction mixture to disperse the reactions to facilitate agitation of the reaction mixture. When solvent is added, the amount of solvent relative to ethylene carbonate is in one aspect up to 1:1.5 w/w. Suitable solvents include an ethereal solvent, a halogenated solvent, a polar aprotic solvent such as acetonitrile or dipolar aprotic solvent such dimethyl formamide or dimethyl sulfoxide. In one aspect the solvent is acetonitrile. The reaction is carried out at room temperature or elevated temperatures, e.g., temperatures between 50° C.-150° C., 80° C.-120° C. or 100° C.

In one aspect, succinimide is added to an 80% solution of ethylene carbonate in acetonitrile (80/20 ethylene carbonate/acetonitrile w/w) and a catalytic amount of DBU. This mixture is heated to 100° C. In another aspect, succinimide is added to an 80% solution of ethylene carbonate in acetonitrile (80/20 ethylene carbonate/acetonitrile w/w) and heated to 95° C. A catalytic amount of DBU is then added and the reaction temperature maintained at 95° C. for a period of 4-5 hours. The temperature is then raised to 105° over two hours and maintained at this temperature until the reaction is complete. In yet another aspect, succinimide is combined with toluene and a catalytic amount of DBU and heated to 100° C. An 80% solution of ethylene carbonate in acetonitrile (80/20 ethylene carbonate/acetonitrile w/w) is added over a period of 4-6 hours and held at 100° C. until the reaction is complete. One volume of toluene relative to ethylene carbonate v/w is used. Toluene is distilled off after the reaction. Roughly, equimolar amounts of succinimide and ethylene carbonate and a catalytic amount of DBU are used; for example 1.06 equivalents of succinimide, 1.0 equivalents of ethylene carbonate and 0.02 equivalents of DBU.

In a second improved process for preparing diroximel fumarate, the monomethyl fumarate starting material can be prepared by: a) reacting methanol and a maleic anhydride starting material represented by:

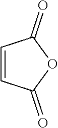

to form an intermediate product represented by

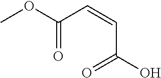

(monomethyl maleate); b) reacting the intermediate product with thionyl chloride to form monomethyl fumarate. The monomethyl fumarate is then reacted in step c) with hydroxyethylsuccinimide in the presence of N-(3-dimethylaminopropyl)-N′-ethylcarbodiimide or a salt thereof (e.g., N-(3-dimethylaminopropyl)-N′-ethylcarbodiimide hydrochloride) and a basic catalyst to form diroximel fumarate. Advantageously, step a) and the reaction in step b) are carried out in one pot, i.e., without isolation of the intermediate product.

The reaction in step b) utilizes between 0.02 and 0.10 molar equivalents of thionyl chloride relative to maleic anhydride. In one aspect, between 0.02 and 0.05 molar equivalents of thionyl chloride relative to maleic anhydride are used; alternatively between 0.04 and 0.06 molar equivalents of thionyl chloride relative to maleic anhydride are used; in another alternative, between 0.04 and 0.05 molar equivalents of thionyl chloride relative to maleic anhydride are used; and in yet another alternative, 0.05 molar equivalents of thionyl chloride relative to maleic anhydride are used. The first reaction is carried out in any suitable solvent, such as ethereal solvents, polar aprotic solvents such as acetonitrile or aromatic solvents such as toluene or xylene. In one aspect, toluene is used as solvent.

In step c), diroximel fumarate is prepared by reacting hydroxyethylsuccinimide with monomethyl fumarate in the presence of N-(3-dimethylaminopropyl)-N′-ethylcarbodiimide or a salt thereof (e.g., N-(3-dimethylaminopropyl)-N′-ethylcarbodiimide hydrochloride) and a basic catalyst. As described above, the basic catalyst is in one aspect is a non-productive nucleophilic catalyst, such as dimethylaminopyridine, 1-methylimidazole and the like. Suitable solvents for this reaction include ketone solvents, ethereal solvents or polar aprotic solvent; in one aspect the solvent used is acetone.

Hydroxyethyl succinimide

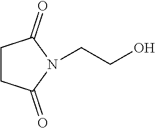

is prepared by reacting succinic anhydride with hydroxyethylamine. The reaction is carried out at a sufficient temperature and for a sufficient length of time to form an intermediate represented by the following structural formula:

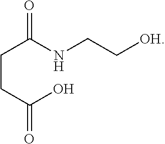 [SMILES: O=C(O)CCC(=O)NCCO]

In one aspect, the temperature is between 45° C. and 70° C., alternatively between 55° C. and 60° C., and the reaction time is one to two hours. After formation of the intermediate, it is reacted with a non-nucleophilic amine base to effect ring closure. The reaction with the non-nucleophilic base is preferably carried out without isolation of the intermediate, i.e., the non-nucleophilic base is typically simply added to the reaction mixture after formation of the intermediate. Suitable non-nucleophilic bases are as described above, and, in one aspect, is diisopropylethylamine. The amount of non-nucleophilic base in one aspect is less than one equivalent relative to succinic anhydride and is preferably catalytic. In one aspect, acetic acid is added to the reaction between the intermediate and the non-nucleophilic amine base to accelerate the ring closure. In one aspect, the acetic acid is added to the reaction after the non-nucleophilic amine and intermediate has been allowed to react for a period of time, for example, up to three hours. In one aspect, between 0.1 and 0.3 equivalents of acetic acid relative to succinic anhydride are used; alternatively, between 0.1 and 0.2 equivalents are used. Any suitable reaction solvent can be used, including alcoholic solvents, ethereal solvent and polar aprotic solvent such as acetonitrile. In one aspect, the reaction solvent is 2-butanol.

The invention is illustrated by the following examples, which are not intended to be limiting in any way.

### EXEMPLIFICATION

### Example 1—Preparation of Monomethyl Fumarate Minimizing Fumaric Acid By-Product

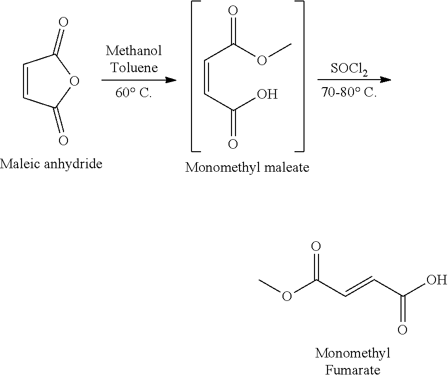

Fumaric acid is the primary impurity in this reaction. Carryover of fumaric acid to the coupling reaction described in Example 5 results in formation of bis(2-(2,5-dioxopyrrolidin-1-yl)ethyl) fumarate impurity:

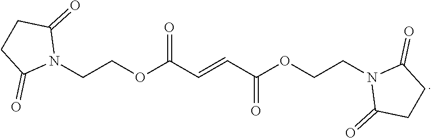 [SMILES: O=C(/C=C/C(=O)OCCN1C(=O)CCC1=O)OCCN1C(=O)CCC1=O]

It has been found that the amount of fumaric acid formed can be controlled by the amount of thionyl chloride used, as shown in Table 1 below:

TABLE 1
Effect of Thionyl Chloride Mol %

| | | | | | | |
|---|---|---|---|---|---|---|
| Exp. | 100 | 100 | 100 | 100 | 40 | 40 |
| Scale | | | | | | |
| (MA | | | | | | |
| input in | | | | | | |
| grams) | | | | | | |
| Thionyl | 2 | 3 | 4 | 5 | 6 | 10 |
| Chloride, | | | | | | |
| mole % | | | | | | |
| Isolated | 75.4 | 80.9 | 81.8 | 80.5 | 77.2 | 74.1 |
| Yield | | | | | | |
| (%) | | | | | | |
| Fumaric | 0.93 | 0.65 | 0.18 | 0.12 | 0.14 | 0.14 |
| acid, | | | | | | |
| % a/a | | | | | | |

Highest yield and lowest fumaric acid content were obtained with 4-5 mole % thionyl chloride. No further gains were realized in term of controlling fumaric acid and increasing yield were observed beyond 5 mole % thionyl chloride usage.

TABLE 2
Comparison of plant results for typical production batches

| | | Thionyl | | % Fumaric Acid impurity in 3 | | |
|---|---|---|---|---|---|---|
| Plant | Batch Size | chloride usage | | representative batches | | |
| Plant I | 625 kg | 2.5 | mole % | 0.7% | 1.0% | 0.9% |
| Plant II | 374 kg | 5 | mole % | 0.14% | 0.15% | 0.17% |

### Example 2—Preparation of Monomethyl Fumarate

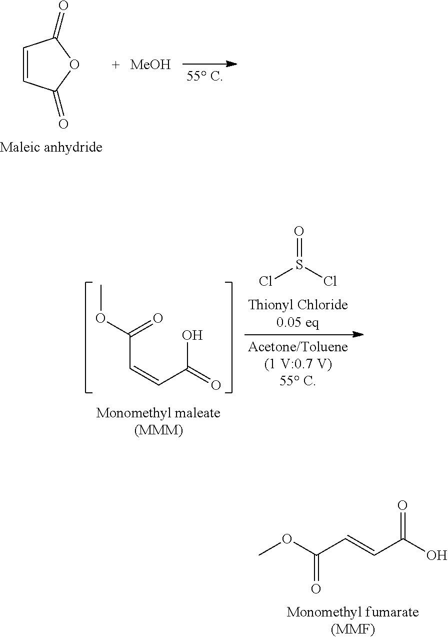

A reactor was charged with maleic anhydride (1.00 eq, 60 g) at 20°° C. to 25° C., and then anhydrous methanol (1.20 eq, 23.53 g) was added in one portion. The resulting mixture was heated to 55° C. with stirring. The reaction was monitored by ¹H-NMR until conversion of maleic anhydride to monomethyl maleate was greater than 96%. Excess methanol was removed under vacuum at 55° C. for 30 minutes, then toluene (30 mL) was added and also removed under vacuum at 55° C. for 30 minutes. Without further isolation or purification of the clear-to-pale yellow thick liquid monomethyl maleate, followed by adding 1 volume of acetone and 0.7 volume of toluene, thionyl chloride (0.05 eq, 3.64 g) was then added dropwise to the reactor at 40° C. to 45° C., and the resulting solution was heated to 55° C. and monitored by HPLC until the monomethyl maleate content was less than 1%, at about 6 hour. Monomethyl fumarate (MMF) precipitated during the reaction. Toluene (138 mL) was added at 55° C. over a 1 hour period and then the reaction mixture was cooled to 40° C. for a 1 hour period. Water (15 mL) was added dropwise to quench the reaction, and the resulting suspension was cooled to 0°° C. over a 3 hour period and held at 0° C. for an additional 2 hour period as the product crystallized from the reaction mixture.

The suspension was filtered and the filter cake was washed with a precooled (0° C.) 40:60 v/v mixture of isopropyl alcohol and water (90 mL) and the cake was dried under vacuum at 40° C. for a 12 h period to obtain 68.61 g of the title product as a fluffy white solid.

### Example 3—Preparation of Hydroxyethyl Succinimide With Improved Conversion Rate

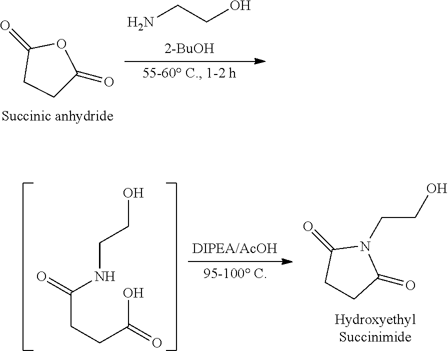

The ring-closure reaction in the presence of diisopropylethylamine (DIPEA) base appears to slow down over time (Table 3). Investigation reveals that reducing the pH by adding acetic acid improves the rate of conversion, as shown in Table 4.

TABLE 3
pH increase during reaction resulted in reduction
of conversion rate (without Acetic acid)

| Time after DIPEA | | Conversion |
|---|---|---|
| addition (h) | pH | (% a/a) |
| 0 | 5.24 | 0.45 |
| 3 | 5.44 | 28.9 |
| 18 | 8.60 | 77.9 |
| 26 | 9.26 | 79.6 |
| 42 | 9.61 | 79.2 |
| 71 | 9.86 | 80.4 |

TABLE 4
Improved reaction rate by adding acetic acid (AcOH)

| Time after DIPEA | Cumulative amount of | | Conversion |
|---|---|---|---|
| addition (h) | AcOH added (mol %) | pH | (% a/a) |
| 0 | 0 | 5.93 | 0.29 |
| 3 | 0 | 6.09 | 28.3 |
| 6 | 0.045 | 6.07 | 47.0 |
| 9 | 0.090 | 5.89 | 60.5 |
| 12 | 0.14 | 5.96 | 68.8 |
| 13 | 0.15 | 5.95 | 71.4 |
| 16 | 0.20 | 6.12 | 78.3 |
| 19 | 0.20 | 6.20 | 82.9 |
| 22 | 0.20 | 6.27 | 85.6 |
| 36 | 0.20 | 6.33 | 94.9 |

### Example 4—Preparation of Hydroxyethyl Succinimide

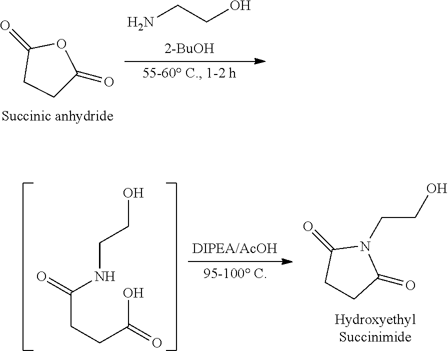

325 kg Succinic anhydride and 1845 kg 2-butanol were heated in a reactor to 55° C. 182 kg monoethanolamine were then added over about 1 hour and stirred for about 30 minutes followed by addition of 126 kg diisopropylethyl amine (DIPEA). The reactor contents were heated to 95-100° C. and stirred for at least 3 hours; 39 kg acetic acid (AcOH) were added and stirring continued for an additional 39 hours until an in process check indicated less than 1.5% a/a succinic anhydride remaining. Approximately 660 liter 2-butanol were then removed by vacuum distillation while maintaining the temperature not to exceed 60° C. 325 L 2-butanol were added to the reactor and the vacuum distillation was repeated to collect approximately 325 L additional distillate. The water content was checked and the process of adding and removing 2-butanol repeated if the water content was greater than 0.5%. The reactor was cooled to 40-60° C. and then 471 L heptane was added. The reactor contents were cooled to 35° C. and 0.33 kg of seed crystals were then added. Stirring was continued and the reactor contents further cooled to −8° C. The product slurry was filtered and washed with 877 L of a mixture of n-heptane/2-BuOH 7:3 v/v and then dried under vacuum at a maximum temperature of 35° C.

### Example 5—Preparation of 2-(2,5-dioxopyrrolidin-1-yl)ethyl methyl fumarate:

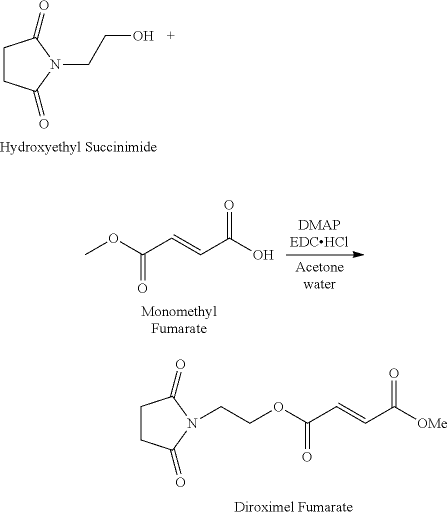

A jacketed reactor was charged with acetone (237 mL), hydroxyethyl succinimide (HES) (1.00 eq, 78.9 g), monomethyl fumarate (MMF) (1.08 eq, 77.4 g), and 4-dimethylaminopyridine (DMAP) (1.5 mol %, 1.02 g. 0.015 eq) while the temperature was maintained at 10° C. to 30° C. Next, N-(3-dimethylaminopropyl)-N′-ethylcarbodiimide hydrochloride (EDC-HCI) (1.13 equiv, 119 g) was added in portions over a 2 hour period while maintaining the temperature at 15° C. to 35° C. The reaction was monitored by HPLC until greater than 97% conversion to the named product was achieved.

The reaction mixture was cooled to 4° C. over a 1 hour period, and water (331 mL) was added over the course of 50-60 min while maintaining the temperature at 4° C. The suspension was further cooled to-5° C. to 5° C. and then filtered. The filter cake was washed with a 80:20 v/v mixture of water and acetone (316 mL) and dried under vacuum at 40° C. to 60°° C. for at least 2 hour to yield the title product.

### Example 6—Preparation of Diroximel fumarate: 2-(2,5-dioxopyrrolidin-1-yl)ethyl methyl fumarate (Vumerity)

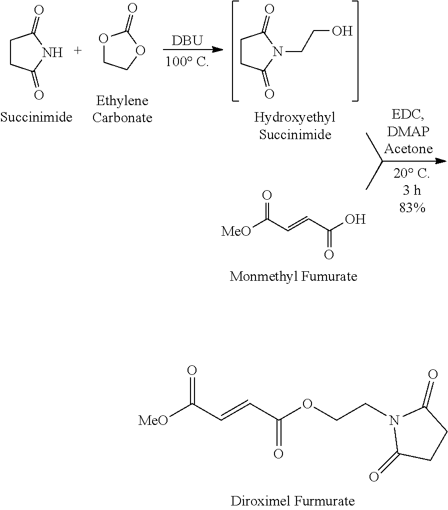

A reactor was charged with acetonitrile (270 mL), ethylene carbonate (1.00 equiv, 270 g), succinimide (1.06 eq, 321 g) and heated to 70° C. with good agitation. 1,8-Diazabicyclo[5.4.0]undec-7-ene (DBU) (0.02 equiv, 9.34 g) was then added to the batch. The batch was then heated to 100°° C. and maintained at this temperature until reaction was complete forming hydroxyethyl succinimide (HES).

The batch was cooled to 50° C., acetone (853 g) is charged and further cooled to about 40° C. With good agitation, DMAP (0.015 equiv, 5.67g) and monomethyl fumarate (MMF) (1.08 equiv, 432 g) were added. N-(3-Dimethylaminopropyl)-N′-ethylcarbodiimide hydrochloride (EDC-HCI) (1.13 equiv, 664 g) was added portion-wise over 2 hours while maintaining the temperature at less than 45° C. The reaction progress was monitored by HPLC. After the reaction is complete, water (27 g) was added and held at 40° C. for 30 minutes followed by the addition of isopropyl alcohol (942 g). The batch was heated to 65° C. to give a clear solution. The batch was then cooled to 0-10° C., filtered and washed with a mixture of isopropyl alcohol and water. The wet cake was dried under vacuum at 50°° C. until dry to afford 678 g (molar yield: 83%) of white title compound.

What is claimed is:

1. A method of preparing a product compound represented by the following structural formula: 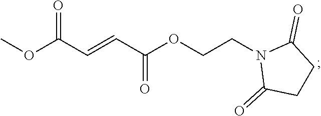 [SMILES: COC(=O)/C=C/C(=O)OCCN1C(=O)CCC1=O]
comprising reacting ethylene carbonate with succinimide in the presence of a base to form a hydroxyethyl succinimide intermediate represented the following structural formula: 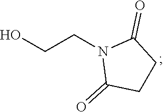
and reacting the intermediate with monomethyl fumarate 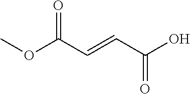
to form the product compound.

2. The method of claim 1, wherein the intermediate is reacted with monomethyl fumarate without isolating the intermediate.

3. The method of claim 1 or 2, wherein the intermediate is reacted with monomethyl fumarate in the presence of a carboxylic acid coupling agent and a basic catalyst.

4. The method of any one of claims 1-3, wherein the carboxylic acid coupling agent selected from a carbodiimide, a phosphonium reagent, an aminium/uranium-imonium reagent, N-ethoxycarbonyl-2-ethoxy-1,2-dihydroquinoline, 2-propanephosphonic acid anhydride, 4-(4,6-dimethoxy-1,3,5-triazin-2-yl)-4-methylmorpholinium salt, bis-trichloromethylcarbonate and 1,1′-carbonyldiimidazole.

5. The method of claim 4, wherein the carboxylic acid coupling agent is a carbodiimide.

6. The method of claim 5, wherein the carbodimide is N-(3-dimethylaminopropyl)-N′-ethylcarbodiimide or a salt thereof.

7. The method of claim 5, wherein the carbodimide is N-(3-dimethylaminopropyl)-N′-ethylcarbodiimide hydrochloride.

8. The method of claim 7, wherein the catalytic base is an amine base.

9. The method of claim 8, the catalytic base is dimethylaminopyridine or 1-methylimidazole.

10. The method of any one of claims 1-9, wherein the reaction between ethylene carbonate and succinimide is carried out in the presence of a base.

11. The method of claim 10, wherein the base is a non-nucleophilic amine base.

12. The method of claim 11, wherein the non-nucleophilic amine base is diazabicyclo[5.4.0]undec-7-ene (DBU).

13. A method of preparing a product compound represented by the following structural formula: 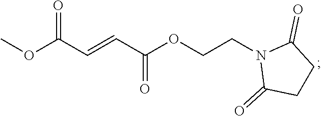 [SMILES: COC(=O)/C=C/C(=O)OCCN1C(=O)CCC1=O]
comprising reacting hydroxyethyl succinimide represented the following structural formula: 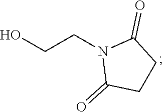
with monomethyl fumarate represented by the following structural formula: 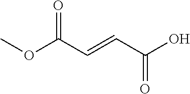
in the presence of N-(3-dimethylaminopropyl)-N′-ethylcarbodiimide or a salt thereof, e.g., N-(3-dimethylaminopropyl)-N′-ethylcarbodiimide hydrochloride, and a basic catalyst to form the product compound.

14. The method of claim 13, wherein the catalytic base is dimethylaminopyridine or 1-methylimidazole.

15. The method of claim 14 or 15, wherein the reaction is carried out in acetone.

16. A method of preparing a product compound represented by the following structural formula: 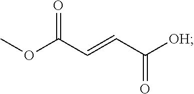
comprising the steps of:
a) reacting methanol and a starting material represented by: 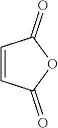
to form an intermediate product represented by 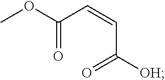
and
b) reacting the intermediate product with thionyl chloride to form the product compound.

17. The method of claim 16, wherein the reaction in step b) is carried out without isolation of the intermediate product.

18. The method of claim 16 or 17, wherein the first reaction is carried out in toluene as solvent.

19. The method of claim 16, 17 or 18, wherein between 0.02 and 0.10 molar equivalents of thionyl chloride relative to starting material are used, for example, between 0.04 and 0.06 molar equivalents of thionyl chloride relative to starting material, for example 0.05 molar equivalents of thionyl chloride relative to starting material.

20. A method of preparing a product compound represented by the following structural formula: 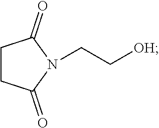
comprising reacting succinic anhydride with hydroxyethylamine to form an intermediate represented by the following structural formula: 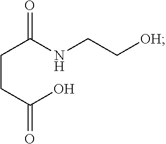 [SMILES: O=C(O)CCC(=O)NCCO]
reacting the intermediate with a non-nucleophilic amine base, such as diisopropylethylamine, preferably without isolation of the intermediate.

21. The method of claim 20, wherein the reaction is carried out in an alcoholic solvent such as 2-butanol.

22. The method of claim 20 or 21, wherein acetic acid is added to the reaction between the intermediate and the non-nucleophilic base.

23. The method of any one of claims 20-22, wherein between 0.1 to 0.3 molar equivalents of acetic acid relative to succinic anhydride is added, for example, between 0.1 to 0.2 molar equivalents of acetic acid relative to succinic anhydride.
